(19) United States
(12) Patent Application Publication
(10) Pub. No.: US 2012/0035689 A1
(43) Pub. Date: Feb. 9, 2012

(54) HEAT AND LIGHT-EMITTING PAD

(75) Inventor: Craig Turtzo, Tarpon Springs, FL (US)

(73) Assignee: ANODYNE THERAPY, L.L.C., Tampa, FL (US)

(21) Appl. No.: 12/849,833

(22) Filed: Aug. 4, 2010

(51) Int. Cl. A61N 5/06; A61B 18/04

(52) U.S. Cl. 607/88; 607/100

(57) ABSTRACT

An application for a heat and light emitting pad has an array of light and heat emitting devices such as LEDS mounted on a circuit board and held within a sponge material. A controller provides a controlled amount of power to the LEDS such that the LEDS emit a therapeutic amount of heat and light for the treatment of pain. The heat and light emitting pad is a thin sheet for insertion between a person and a surface on which that person rests.

### CROSS-REFERENCE TO RELATED APPLICATION

This application is related to U.S. patent application titled “SYSTEM, METHOD AND APPARATUS FOR TREATING BACK PAIN DURING REST,” attorney docket 552.4 filed even date here within. This application is also related to U.S. patent application titled “INTEGRATED SYSTEM, METHOD AND APPARATUS FOR TREATING BACK PAIN DURING REST,” attorney docket 552.6 filed even date here within. This application is also related to U.S. patent application titled “WORN HEAT AND LIGHT-EMITTING DEVICE,” attorney docket 552.7 filed even date here within.

### FIELD

This invention relates to the field of pain relief and more particularly to a system for the deliver of heat and infrared light to a user for the relief of pain.

### BACKGROUND

It is known that exposure to heat and certain wavelengths of light are useful to temporarily increase local circulation, reduce pain and to enhance healing as detailed in Michlovitz and Nolan, Modalities for Therapeutic Intervention (4th Ed.), F.A. Davis Company (2005). Recent inventions have used light and/or heat as a therapeutic device for the relief of pain. In particular, it has been shown that infra-red and near infra-red light of certain wavelengths possess therapeutic qualities. Exposure to certain wavelengths of light is known to alleviate various effects that sun exposure, gravity, pollution and chemicals have on the skin.

LED Devices that emit infrared wavelengths of light are well known and are capable of providing sufficient light for therapeutic effects to persons exposed to the light under certain conditions. Additionally, such LED Devices also emit heat, which also provide therapeutic effects such as reducing pain in certain situations. Existing stand-alone LED devices do not provide for sufficiently convenient at-home applications of infrared light and heat. For example, cable-connected devices are available having multiple LED arrays for the irradiation of a user's leg or foot, but the cable causes problems as the user moves. Likewise, battery powered devices also having multiple LED arrays for the irradiation of a user's foot or leg improve upon this problem, but the batteries cause an issue by presenting hard bulges that exert pressure on the user when the user rests in certain positions.

What is needed is a system that will irradiate a locale of a user with heat and light while the user rests.

### SUMMARY OF THE INVENTION

The present invention includes a system, method and apparatus for directing light and heat towards an area of pain of a person while the person rests on a section of furniture (e.g., sleeps on a mattress). The light and heat is provided by an array of LEDS that are arranged to form a sheet that is thin enough to, preferably, fit comfortably between a person and, for example, a bed or other furniture.

In one embodiment, a treatment pad for providing heat and light to a person for the purpose of treating pain is disclosed. The treatment pad includes an array of LEDS mounted to a circuit board and held by a bendable sponge material. A controller is electrically interfaced to the LEDS and selectively provides power to the LEDS. A power switch is electrically interfaced to the controller, instructing the controller to start and stop providing power to the LEDS. A source of electrical power is connected to the controller.

In another embodiment, a method of reducing pain in a person is disclosed including providing a treatment pad that includes an array of LEDS mounted to a circuit board and held by a bendable sponge material with a controller electrically interfaced to the LEDS, selectively providing power to the LEDS. A power switch is electrically interfaced to the controller for instructing the controller to start and stop providing power to the LEDS and a source of electrical power connected to the controller. The method continues with placing the treatment pad on a surface with the array of LEDS facing away from the surface then resting on the treatment pad by a person for a period of time, during which the array of LEDS emit light and heat.

In another embodiment, an apparatus for providing heat and light to a person for the purpose of treating pain is disclosed including a device for emitting both heat and light, the device forming a sheet. Also included is a device for controlling power to the device for emitting both the heat and the light and a device for initiating and stopping the power. The devices are enclosed in a material such as cloth and connected to a source of electrical power.

### BRIEF DESCRIPTION OF THE DRAWINGS

The invention can be best understood by those having ordinary skill in the art by reference to the following detailed description when considered in conjunction with the accompanying drawings in which:

FIG. 1 illustrates a perspective view of a first embodiment.

FIG. 2 illustrates a second perspective view of a second embodiment.

FIG. 3 illustrates a third perspective view of the second embodiment.

FIG. 4 illustrates a fourth perspective view of the second embodiment.

FIG. 5 illustrates a cross-sectional view of the second embodiment.

FIG. 6 illustrates a second cross-sectional view of the second embodiment.

FIG. 7 illustrates a third cross-sectional view of the second embodiment.

FIG. 8 illustrates a perspective view of a third embodiment.

FIG. 9 illustrates a first cross-sectional view of the second embodiment in use.

FIG. 10 illustrates a first cross-sectional view of the second embodiment in use.

FIG. 11 illustrates a perspective view of a fourth embodiment.

FIG. 12 illustrates a perspective view of a fourth embodiment in use.

FIG. 13 illustrates a schematic view of an exemplary control system of all embodiments.

FIG. 14 illustrates a cutaway side plan view of an exemplary LED array and control system of all embodiments.

FIG. 15 illustrates a top plan view of an exemplary LED array and control system of all embodiments.

FIG. 16 illustrates a cutaway side plan view of an exemplary LED array and control system of the first embodiment.

### DETAILED DESCRIPTION

Reference will now be made in detail to the presently preferred embodiments, examples of which are illustrated in the accompanying drawings. Throughout the following detailed description, the same reference numerals refer to the same elements in all figures.

Throughout this description, a mattress (bed) is used as an example of furniture. This is but an example and it is anticipated that the present method, apparatus and system be used in conjunction and/or integrated into any type of human-supporting or contact furniture such as pillows, chairs, recliners, couches, sofas, futons, car/vehicle seats etc. It is further anticipated that the present method, apparatus and system be applied to other devices/systems that normally contact the human body such as bicycle seats, motorcycle seats, arm rests, head rests, etc. When integrated into, for example, a pillow, the pillow is, for example, placed behind the back when the person is sitting in a chair for back pain or against the neck for neck pain.

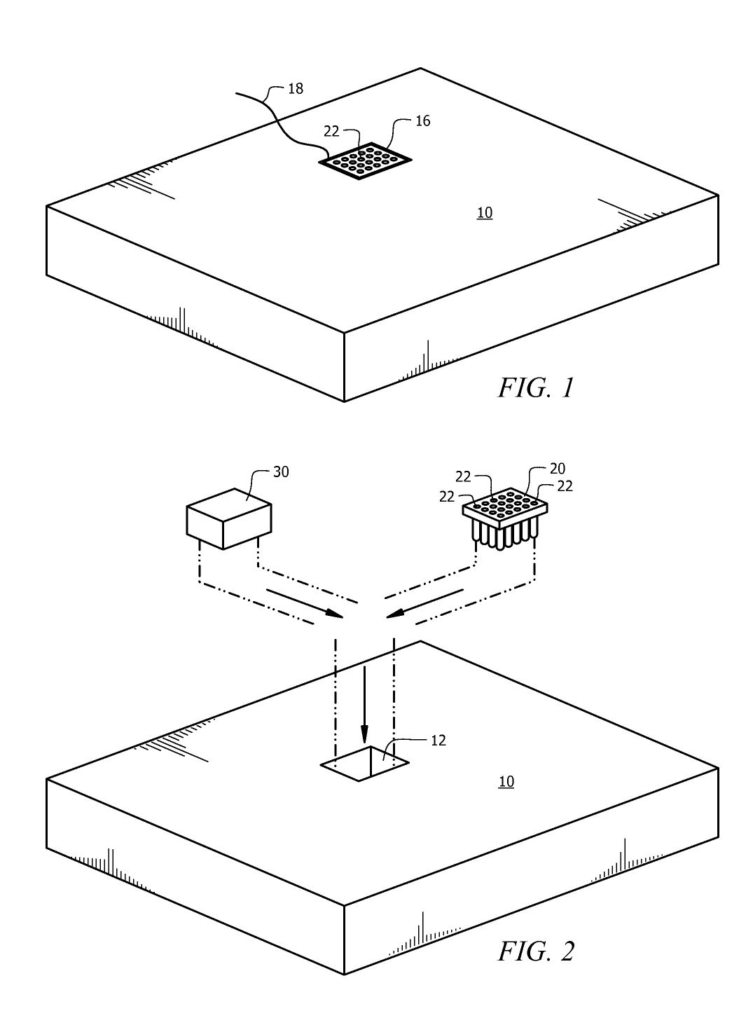

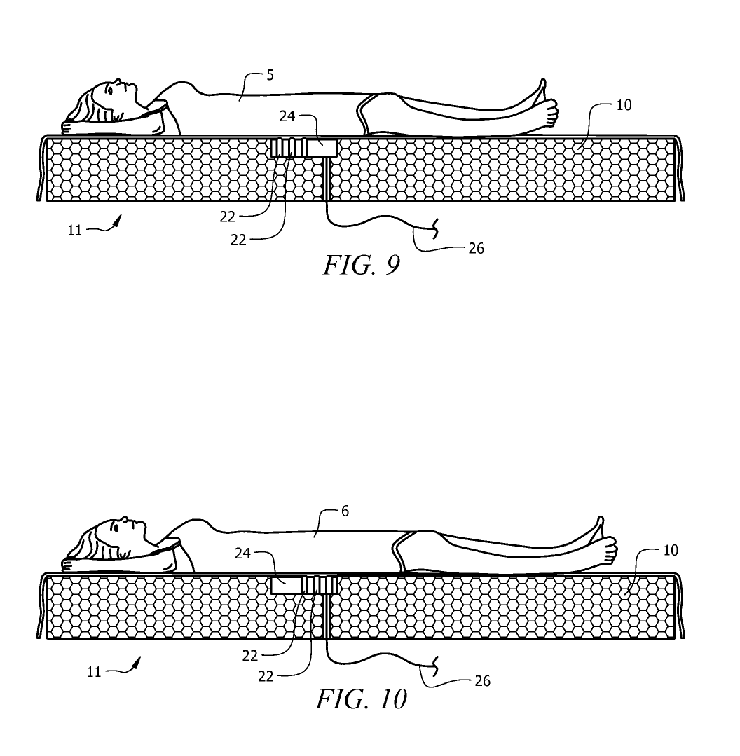

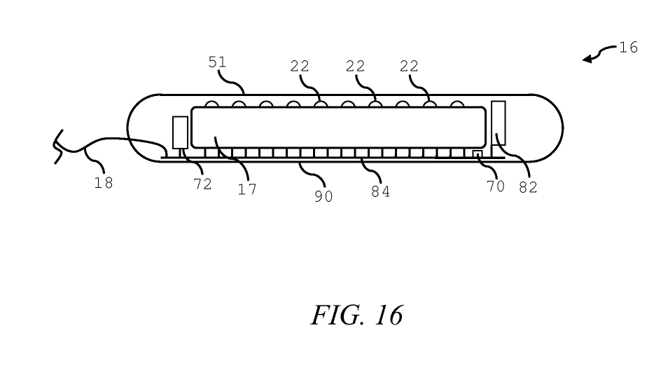

Referring to FIG. 1, a perspective view of a first embodiment is shown. In this embodiment, an array of LEDS 22 is integrated into a treatment pad 16 that is positioned on, for example, a bed/mattress 10. The LEDS 22 are connected by a circuit board 84 and held in a soft, rubber/plastic holder 17 (see FIG. 16). In some embodiments, the thin pad 16, LEDS 22 and circuit board 84 are enclosed in a cloth cover for protection. The LEDS are powered through a cable 18 connected to a power supply/controller (see FIG. 13). The power supply/controller provides a controlled amount of electrical energy to the array of LEDS 22, causing the LEDS 22 to emit light at one or more wavelengths, preferably including infrared or near infrared. Additionally, the LEDS 22 produce heat. Both the light and heat provide therapeutic effect to a person in contact with the thin pad 16 of LEDS 22 when in contact with the person. It is anticipated that, for relief of back pain, the person positions themselves such that the person's back or lower back is located directly over the thin pad 16 of LEDS 22 (see FIGS. 9 and 10). The invention is anticipated to perform equally as well with other parts of the human body.

Referring to FIG. 2, a second perspective view of a second embodiment is shown. This embodiment includes a modular section within furniture such as a mattress 10. In such, a cavity 12 is made/formed/left in the furniture/mattress 10 into which an option module 30/20 is fit. In such, for deployment of the furniture/mattress 10 without the array 20 of LEDS 22, a blank insert 30 is placed in the cavity. Therefore, when the furniture/mattress 10 is covered (e.g., with a sheet 16—see FIG. 7) and the cavity 12 is filled with the blank insert, it is difficult to detect by a person using the furniture/mattress 10. Alternately, when the array 20 of LEDS 22 is to be used, the blank insert 30 is removed and the array 20 of LEDS 22 is inserted into the cavity 12 of the mattress/furniture 10. As shown in FIG. 2, the entire array 20 is populated with LEDS 22, preferably infrared or near-infrared LEDS 22 or a mixture of infrared or near-infrared LEDS 22 and/or visible light LEDS 22. Any wavelength of LEDS 22 is anticipated. In embodiments in which the furniture/mattress 10 is a mattress 10, it is anticipated that the mattress 10 comprise any known mattress material currently used in the industry, including foam rubber (e.g., latex foam), memory foam (visco-elastic memory foam material) and the like. Memory foam material is often made from synthetic polyurethane foam material with the addition of certain types of chemicals which increase the weight or density of the foam, as known in the industry. It is further anticipated that the mattress 10, in some embodiments, is an air mattress 10 and, therefore, the mattress 10 is completely sealed and air-tight around the cavity 12.

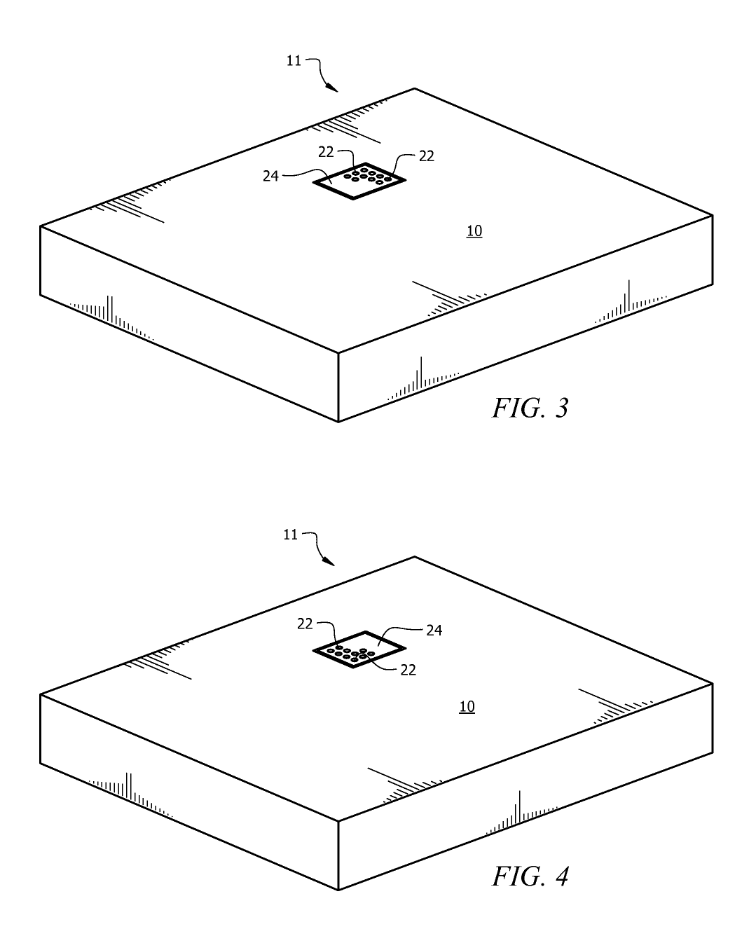

Referring to FIGS. 3 and 4, a third and fourth perspective view of the second embodiment is shown. In this the array 24 of LEDS 22 is populated with LEDS 22 towards one end of the array 24 and the array 24 is passive at the opposite end (absent of active LEDS). In such, when a shorter person uses the array 24, for example to relieve lower back pain, the array 24 is positioned in the mattress as shown in FIG. 3 and the active LEDS 22 are positioned toward the head of the bed 11, thereby aligning with the shorter person's lower back. Alternately, as shown in FIG. 4, when a taller person uses the array 24, for example to relieve lower back pain, the array 24 is turned 180 degrees within the mattress so that the active LEDS 22 are positioned away from the head of the bed 11, thereby aligning with the taller person's lower back.

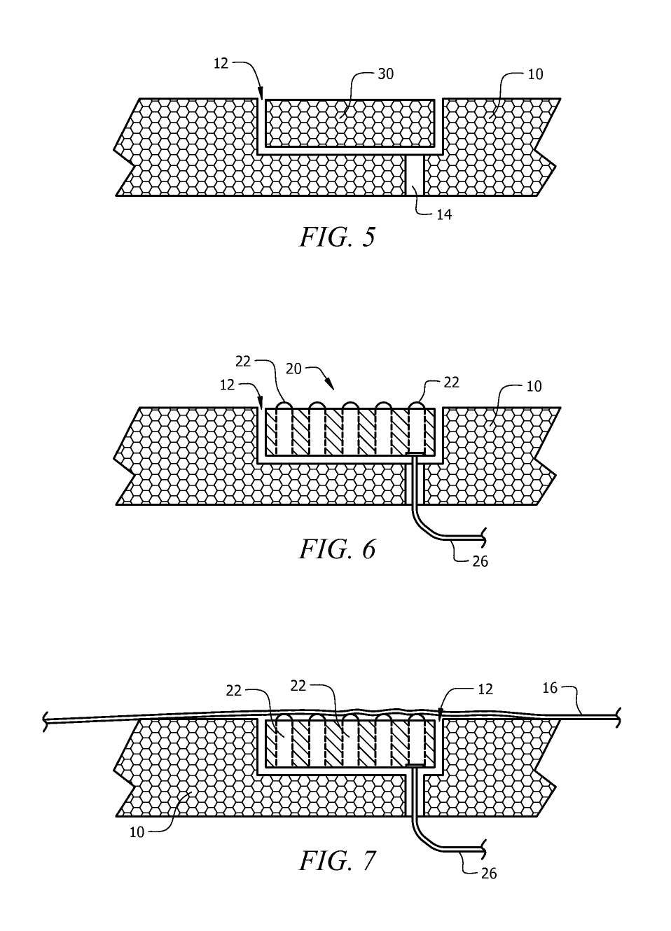

Referring to FIG. 5, a cross-sectional view of the second embodiment is shown. In this view, the blank insert 30 is snuggly fit within the cavity 12 (e.g. a tight fit limiting or reducing movement/sliding along any axis). In some embodiments, a hole or slot 14 connects the cavity with the bottom of the furniture/mattress 10 for running cables 26 (see FIG. 6).

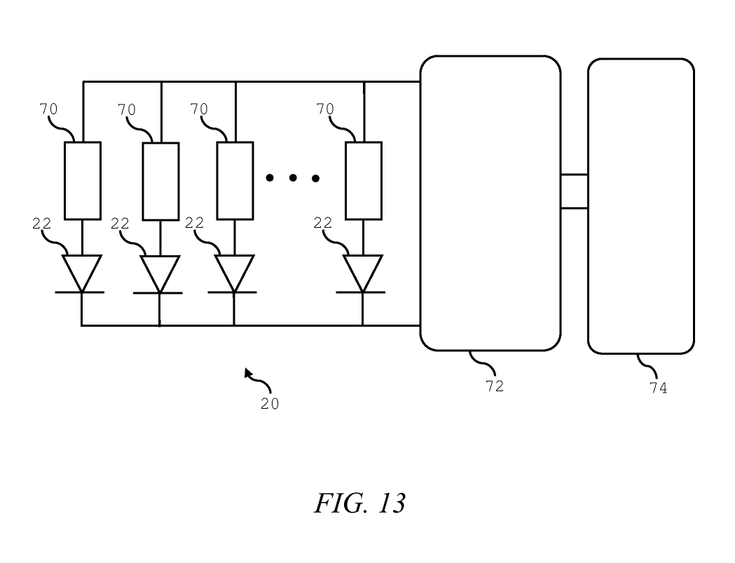

Referring to FIG. 6, a second cross-sectional view of the second embodiment is shown. In this view, the array 20 of LEDS 22 is snuggly fit within the cavity 12 (e.g. a tight fit limiting or reducing movement/sliding along any axis). In some embodiments, an electrical cable 26 from the LEDS 22 to a controller 70 and/or power supply 74 (see FIGS. 12 and 13) exits through the hole or slot 14 connects the cavity with the bottom of the furniture/mattress 10 for running cables 26.

Referring to FIG. 7, a third cross-sectional view of the second embodiment is shown. In this view, the array 20 of LEDS 22 is positioned within the cavity 12. In some embodiments, an electrical cable 26 from the LEDS 22 to a controller 72 and/or power supply 74 (see FIG. 13) exits through the hole or slot 14 that connects the cavity with the bottom of the furniture/mattress 10 for running cables 26. In this view, a cloth cover 16 is positioned over the array 20 of LEDS 22. Studies have shown that by increasing the power and/or duration of activity of the LEDS 22, sufficient heat and light penetrate layers of cloth 16 such as sheets, bedding, etc, and provide relief for pain such as lower back pain. Therefore, as shown in FIG. 7, the sheet 16 (for example) covers the LEDS 22 and the user positions their body such that the area in pain rests on top of the sheet 16 where the LEDS 22 lay beneath.

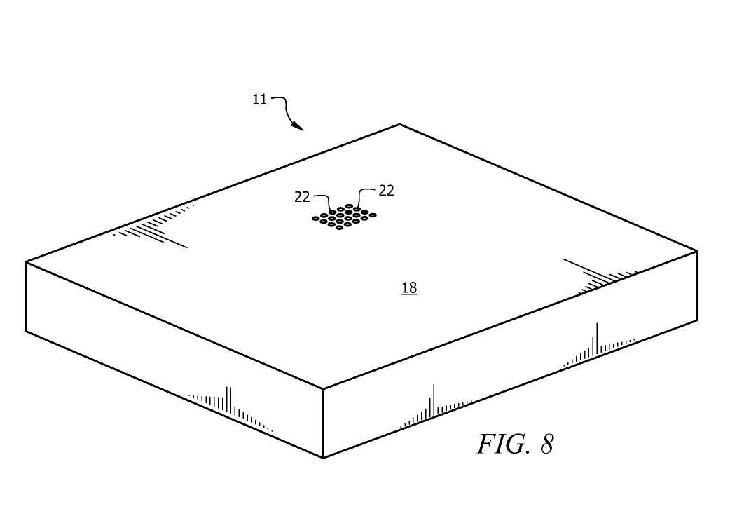

Referring to FIG. 8, a perspective view of a third embodiment is shown. In this embodiment, a set of LEDS 22 are integrated directly into furniture/mattress 18. The LEDS 22 are fitted into holes or apertures of the mattress 18 and are in a fixed position relative to the head 11 of the mattress. Again, as in FIG. 7, it is anticipated that in some embodiments, a cloth cover 16 is positioned over the LEDS 22 and the power and/or duration of activity of the LEDS 22 is increased, providing sufficient heat and light to penetrate the layers of cloth 16 such as sheets, bedding, etc, and provide relief for pain such as lower back pain.

Referring to FIGS. 9 and 10, a first and second cross-sectional view of the second embodiment in use is shown. In this the array 24 of LEDS 22 is populated with LEDS 22 towards one end of the array 24 and the array 24 is passive at the opposite end. In such, when a shorter person 5 uses the array 24, for example to relieve lower back pain, the array 24 is positioned in the mattress 10 as shown in FIG. 9 and the active LEDS 22 are positioned toward the head of the bed 11, thereby aligning with the shorter person's 5 lower back. Alternately, as shown in FIG. 10, when a taller person 6 uses the array 24, for example to relieve lower back pain, the array 24 is positioned in the mattress 10 so that the active LEDS 22 are positioned away from the head of the bed 11, thereby aligning with the taller person's 6 lower back.

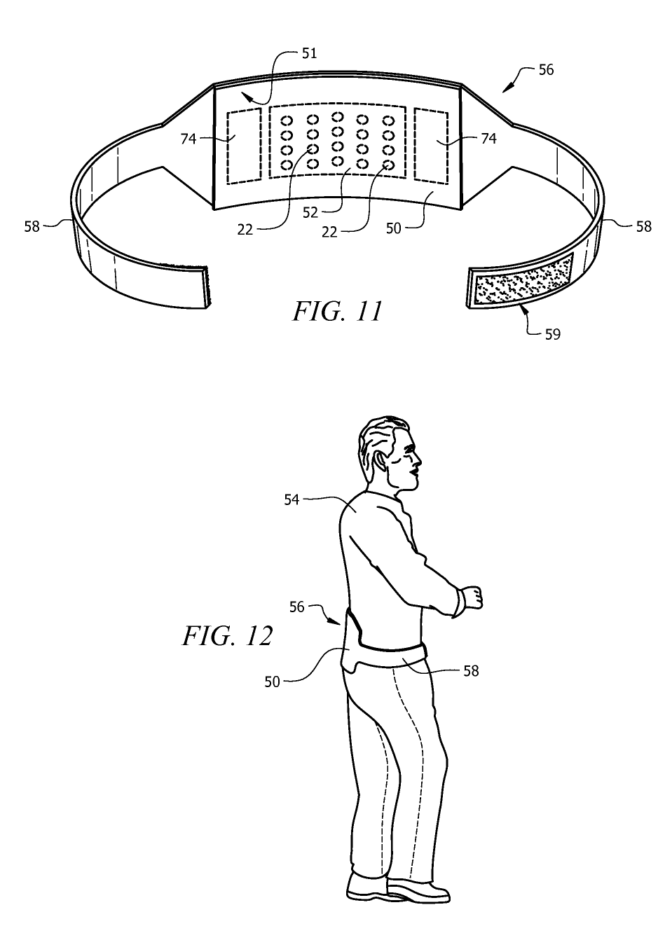

Referring to FIGS. 11 and 12, perspective views of a fourth embodiment is shown. In this embodiment, an array 52 of LEDS 22 and power sources 74 are integrated into a wearable device 56 that attaches to a person's body 54 with a belt 58 and fastener 59. The belt 58 and fastener 59 are any such device as known in the industry and the exemplary hook and loop fastener 59 is one possible fastener. Other belt 58 lengths, widths and shapes are anticipated as well as other fasteners 59 such as buckles, buttons, snaps, etc.

In some embodiments, the array 52 of LEDS 22 and power sources 74 (e.g. batteries 74) are covered with a cloth material 51 such as nylon, silk, polyester, cotton, etc. As previously discussed, the power and/or duration of activity of the LEDS 22 is/are increased, providing sufficient heat and light to penetrate the layer of cloth 51 to provide relief for pain such as lower back pain as shown in FIG. 12. It is anticipated that the wearable device 56 of this embodiment is adaptable in size and shape to be worn on other parts of the body 54 such as feet, ankles, knees, legs, other areas of the back, neck, arms, hands and head. By situating the power source 74 (e.g. batteries 74) within the back area of the wearable device 56, heat emitted from the batteries as they discharge provides additional heat to, for example, the person's 54 back (see FIG. 12).

Referring to FIG. 13, a schematic view of an exemplary control system of all embodiments is shown. In this example, each LED 22 in the array 20 is current limited by an individual resistor 70. To illuminate the LEDS 22, a voltage is supplied by the controller 72 and the value of the resistors 70 determines the current flowing through each LED 22 and hence, the power output of each LED 22. The lower the resistance of each resistor 70, the higher the power output of its corresponding LED 22. This is an example of how such LEDS 22 are provided with a predetermined amount of power and other methods are well known using various combinations of LEDS 22 connected in series and/or parallel with various combinations of resistors 70 or other current controlling devices. In some embodiments, the current is directly controlled by the controller 72, eliminating the need for resistors 70. Any known system for providing a controlled amount of power to the LEDS 22 is anticipated here within.

As discussed previously, in applications in which a cloth 16/51 is situated between the user and the LEDS 22, the power to the LEDS 22 is increased to provide greater heat and light output to overcome the loss inserted by the cloth 16/51. This is accomplished in any way known in the industry including selecting lower resistance values of the resistors 70 or increasing the voltage output of the controller 72, etc.

The controller 72 provides power to the LEDS 22 during an active period. It is anticipated that the controller 72 provide power to the LEDS 22 for a pre-determined period of time from minutes up to continuously, as needed to address the user's specific pain. It is also anticipated that the controller 72 provide any known sequencing of power levels and timing as needed to address the specific pain. For example, for certain pain/healing operations, it is desired to alternate heat/cool and the controller 72 provides power for one period, thereby providing heat, and no or little power for another period, thereby removing the heat. Additionally, the controller 72, in some embodiments, provides pulse width modulation to control the power to the LEDS in which, the greater the pulse width, the greater the power supplied to the LEDS and the greater the light and heat intensity. In this embodiment, the pulse width and frequency is either fixed or variable.

For completeness, a power source 74 is shown, as known in the industry. Any known power source 74 is anticipated, including, but not limited to, a battery pack, a rechargeable battery pack and a power supply such as a power brick for converting household electric power into a DC voltage.

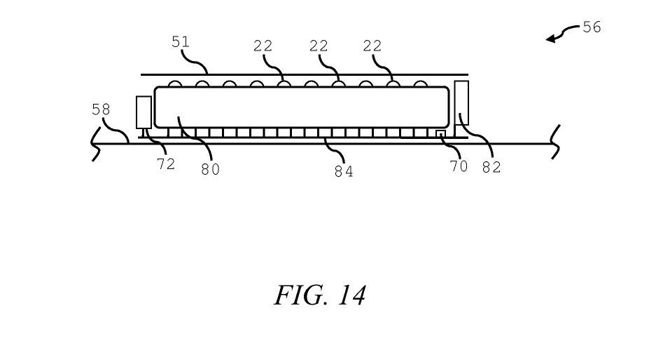

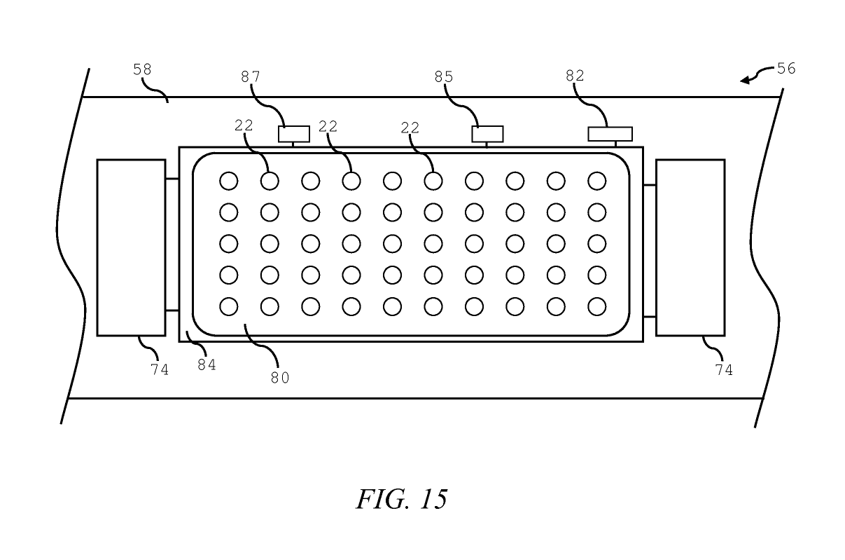

Referring to FIG. 14, a cutaway side plan view of an exemplary LED array control system of all embodiments is shown. In the preferred embodiment, the LEDS 22 are held in holes of a material 80, preferably a soft, sponge-like material that also conducts heat to provide a more even heat should one LED 22 heat more than another LED 22. The LEDs are physically and electrically interfaced to a circuit board 84 situated between the belt 58 and the LEDs 22/material 80. Other components such as the controller 72 and resistors 70 (if needed) are preferably mounted on the circuit board 84. Also connected to the circuit board 84 is a power switch 82 for signaling the controller 72 to enter an operating mode. Responsive to the user operating the power switch 82, the controller provides power to the LEDS 22. For example, after the user operates the power switch 82, the controller 72 provides power to the LEDS 22 for a fixed amount of time and then removes power to the LEDS 22 for another fixed amount of time, repeating this sequence for a pre-determined number of cycles. Although any switch 82 is anticipated, a proximity switch 82 is preferred to reduce the chance of the user inadvertently tripping the switch while wearing the belt 56. The proximity switch 82 preferably has hysteresis requiring the user to touch the proximity switch 82 for a time period before the operating mode is entered and requiring the user to again touch the proximity switch 82 for a time period before shutting off power. To inform the user that the operating mode has been entered or the system is shut off, a sounder 87 (see FIG. 15) is provided in some embodiments, preferably emitting one sound or sequence for entering the operating mode and another for power off.

In some embodiments, the LEDS 22 and power sources 74 (not visible in FIG. 14) are covered with a cloth material 51 such as nylon, silk, polyester, cotton, etc. As previously discussed, the power and/or duration of activity of the LEDS 22 is/are increased, providing sufficient heat and light to penetrate the layer of cloth 51 to provide relief for pain such as lower back pain. It is anticipated that the wearable device 56 of this embodiment is adaptable in size and shape to be worn on other parts of the body 54 such as feet, ankles, knees, legs, other areas of the back, neck, arms, hands and head. By situating the power source 74 (e.g. batteries 74) within the back area of the wearable device 56, heat emitted from the batteries as they discharge provides additional heat to, for example, the person's 54 back (see FIG. 12).

Referring to FIG. 15, a top plan view of an exemplary LED array and control system of all embodiments is shown. In the preferred embodiment, the LEDS 22 are held in holes of a material 80, preferably a soft, sponge-like material that also conducts heat to provide a more even heat should one LED 22 heat more than another LED 22. The LEDs are physically and electrically interfaced to a circuit board 84 situated between the belt 58 and the LEDs 22/material 80. Other components such as the controller 72 and resistors 70 (if needed) are preferably mounted on the circuit board 84. The power source 74 (e.g. batteries 74) is electrically connected to the circuit board 84 the power source 74 (e.g. batteries 74) is situated within the back area of the wearable device 56. In some embodiments, the power source 74 is located near the circuit board 84 so that as the batteries 74 discharge, heat emitted provides additional heat to, for example, the person's 54 back (see FIG. 12).

Also connected to the circuit board 84 is a power switch 82 for signaling the controller 72 to enter an operating mode. Responsive to the user operating the power switch 82, the controller provides power to the LEDS 22. For example, after the user operates the power switch 82, the controller 72 provides power to the LEDS 22 for a fixed amount of time and then removes power to the LEDS 22 for another fixed amount of time, repeating this sequence for a pre-determined number of cycles. Although any switch 82 is anticipated, a proximity switch 82 is preferred to reduce the chance of the user inadvertently tripping the switch while wearing the belt 56. The proximity switch 82 preferably has hysteresis requiring the user to touch the proximity switch 82 for a time period before the operating mode is entered and requiring the user to again touch the proximity switch 82 for a time period before shutting off power. To inform the user that the operating mode has been entered or the system is shut off, a sounder 87 (see FIG. 15) is provided in some embodiments, preferably emitting one sound or sequence for entering the operating mode and another for power off.

The power source 74 is charged through a power connector 85 that is connected to an external power source (not shown) such as a wall-wart as known in the industry.

Referring to FIG. 16, a cutaway side plan view of an exemplary LED array control system of the first embodiment is shown. In the preferred implementation, the LEDS 22 are held in holes of a material 17, preferably a soft, sponge-like material that also conducts heat to provide a more even heat should one LED 22 heat more than another LED 22. The LEDs are physically and electrically interfaced to a circuit board 84. Other components such as the controller 72 and resistors 70 (if needed) are preferably mounted on the circuit board 84. Also connected to the circuit board 84 is a power switch 82 for signaling the controller 72 to enter an operating mode. Responsive to the user operating the power switch 82, the controller provides power to the LEDS 22. For example, after the user operates the power switch 82, the controller 72 provides power to the LEDS 22 for a fixed amount of time and then removes power to the LEDS 22 for another fixed amount of time, repeating this sequence for a pre-determined number of cycles. Although any switch 82 is anticipated, a proximity switch 82 is preferred to reduce the chance of the user inadvertently tripping the switch while wearing the belt 56. The proximity switch 82 preferably has hysteresis requiring the user to touch the proximity switch 82 for a time period before the operating mode is entered and requiring the user to again touch the proximity switch 82 for a time period before shutting off power. To inform the user that the operating mode has been entered or the system is shut off, a sounder 87 (see FIG. 15) is provided in some embodiments, preferably emitting one sound or sequence for entering the operating mode and another for power off.

In some embodiments, the LEDS 22 and electronics 70/72/82/84 are enclosed within a cloth material 90 such as nylon, silk, polyester, cotton, etc. As previously discussed, the power and/or duration of activity of the LEDS 22 is/are increased, providing sufficient heat and light to penetrate the layer of cloth 90 to provide relief for pain such as lower back pain. It is anticipated that any number of LEDS 22 be present to cover a suitable area of the user's body. In some embodiments, power is provided by a power cable 18 connected to an external power source such as a power brick or wall-wart, as known in the industry.

Equivalent elements can be substituted for the ones set forth above such that they perform in substantially the same manner in substantially the same way for achieving substantially the same result.

It is believed that the system and method and many of its attendant advantages will be understood by the foregoing description. It is also believed that it will be apparent that various changes may be made in the form, construction and arrangement of the components thereof without departing from the scope and spirit of the invention or without sacrificing all of its material advantages. The form herein before described being merely exemplary and explanatory embodiment thereof. It is the intention of the following claims to encompass and include such changes.

What is claimed is:

1. A treatment pad for providing heat and light to a person for the purpose of treating pain, the treatment pad comprising:
an array of LEDS mounted to a circuit board, the array of LEDS held by a bendable sponge material;
a controller electrically interfaced to the LEDS, the controller selectively providing power to the LEDS;
a power switch electrically interfaced to the controller, the power switch instructing the controller to provide power to the LEDS and to stop providing power to the LEDS; and
a source of electrical power connected to the controller.

2. The treatment pad of claim 1, wherein the array of LEDS, the controller and the power switch are enclosed in a cloth material.

3. The treatment pad of claim 1, wherein the LEDS emit infrared light.

4. The treatment pad of claim 1, wherein a subset of the LEDS emit infrared light and a subset of the LEDS emit visible light.

5. The treatment pad of claim 2, wherein the power provided to the LEDS by the controller is increased to compensate for heat and light loss caused by the cloth material.

6. The treatment pad of claim 1, wherein the power switch is a proximity switch.

7. A method of reducing pain in a person, the method comprising:
providing a treatment pad comprising:
an array of LEDS mounted to a circuit board, the array of LEDS held by a bendable sponge material;
a controller electrically interfaced to the LEDS, the controller selectively providing power to the LEDS;
a power switch electrically interfaced to the controller, the power switch instructing the controller to provide power to the LEDS and to stop providing power to the LEDS;
a source of electrical power connected to the controller;
placing the treatment pad on a surface, the array of LEDS facing away from the surface;
resting on the treatment pad by the person for a period of time, the array of LEDS emitting light and heat during a portion of the period of time.

8. The method of claim 7, wherein the array of LEDS, the controller and the power switch are enclosed in a cloth material.

9. The method of claim 7, wherein the LEDS emit infrared light.

10. The method of claim 7, wherein a subset of the LEDS emit infrared light and a subset of the LEDS emit visible light.

11. The method of claim 8, wherein the power provided to the LEDS by the controller is increased to compensate for heat and light loss caused by the cloth material.

12. The method of claim 7, wherein the power switch is a proximity switch.

13. The method of claim 7, wherein the period of time is when the person is sleeping and the portion of the period of time is less than or equal to the period of time when the person is sleeping.

14. An apparatus for providing heat and light to a person for the purpose of treating pain, the apparatus comprising:
a means for emitting both heat and light, the means for emitting both heat and light forming a sheet;
a means for controlling power to the means for emitting both the heat and the light;
a means for initiating and stopping the means for controlling power;
a source of electrical power electrically interfaced to the means for controlling power; and
a means for enclosing the means for emitting, the means for controlling power and the means for initiating.

15. The apparatus of claim 14, wherein the light comprises infrared light.

16. The apparatus of claim 14, wherein the light comprises a combination of infrared light and visible light.

17. The apparatus of claim 14, wherein the means for controlling the power provides substantial power to compensate for lost heat and lost light caused by the means for enclosing.

18. The apparatus of claim 14, wherein the means for emitting both the heat and the light is an array of infrared LEDS.

19. The apparatus of claim 14, wherein the means for initiating is a proximity switch.

20. The apparatus of claim 14, wherein the means for enclosing is a cloth material.
